(19) United States
(12) Patent Application Publication
(10) Pub. No.: US 2003/0171543 A1
(43) Pub. Date: Sep. 11, 2003

(54) High throughput mutagenesis screening method

(76) Inventors: Richard R. Bott, Burlingame, CA (US); James T. Kellis JR., Portola Valley, CA (US); Thomas B. Morrison, Winchester, MA (US)

Correspondence Address: JANET KAISER CASTANEDA, GENENCOR INTERNATIONAL, INC., 925 PAGE MILL ROAD, PALO ALTO, CA, 94304-1013, US

(21) Appl. No.: 10/092,227

(22) Filed: Mar. 5, 2002

(51) Int. Cl. G01N 33/53; C12N 15/85; C07K 14/00; C12P 21/02; C12N 5/06

(52) U.S. Cl. 530/350; 435/7.1; 435/320.1; 435/455; 435/325; 435/69.1

(57) ABSTRACT

A high throughput mutagenesis screening method selects sites for saturation scanning using protein structural considerations. Site-saturation libraries are created and screened using assays for sites having protein properties of interest. The sites are categorized for the properties of interest, trends, if any, are identified; and, a variant having a desired property is selected for additional library creation procedures. The additional libraries are created using feedback from the determined categories. One set of additional libraries repeats construction of the previous libraries using the feedback. A second set of additional libraries are new libraries created at sites that are expected to be beneficial based on the feedback. The categories may be coded, using color or other indicia, to allow easy identification of trends.

### FIELD OF THE INVENTION

The invention relates to mutagenesis methods for protein engineering. Specifically, the invention relates to a mutagenesis method which includes feedback adjustment from systematic result evaluation. More specifically, the invention relates to a site-saturation mutagenesis method which screens for variants with one or more desirable protein properties and evaluates screening results to provide feedback for repeat screening and construction of new libraries.

### BACKGROUND OF THE INVENTION

Mutagenesis procedures are known for modifying proteins to obtain desired protein properties. The nucleotide sequence of a cloned gene encoding a protein is mutated and the modified gene is expressed to produce mutants, which may be screened for activities of interest. The mutant properties may be compared with the properties of wild-type protein.

A number of mutagenesis methods are known in the art. One such method is site-saturation mutagenesis which results in a mutated gene population that consists of otherwise identical genes, and a random given codon. The random given code can code for any amino acid, and the position is said to be saturated. (Airaksinen, A and Hovi, T. (1998) Nucleic Acids Research, Vol. 26, No. 2: 576-581). There are several different site-saturation methods.

Because most mutagenesis methods present a great number of amino acid mutation options, screening of a large number of variants generally is required to produce a desired protein property. Generally, screening is repeated over and over to produce a beneficial variant. There is a continuing need in the art for mutagenesis and screening methods having efficient procedures for data analysis to obtain desired protein engineering results.

### SUMMARY OF THE INVENTION

A high throughput mutagenesis screening method selects sites for saturation scanning using protein structural considerations. Site-saturation libraries are created and screened using assays selected to identify clones having protein properties of interest. The mutation sites are categorized for the properties of interest, and trends, if any, are identified. A variant having a desired property is selected for additional library creation procedures. The additional libraries are created using feedback from the determined categories. One set of additional libraries repeats construction of the previous libraries using the feedback. A second set of additional libraries are new libraries created at sites that are expected to be beneficial based on the feedback.

In one aspect of the invention, the categories are coded, using color or other indicia, to allow easy identification of trends.

### BRIEF DESCRIPTION OF THE DRAWINGS

FIG. 1 is a diagrammatic view illustrating the general principle of the invention.

FIG. 2 is a graph showing the relationship between polyesterase activity and protein concentration determined by TCA assay for several different Bacillus subtilis transformants.

FIGS. 3 and 4 are graphs showing the polyesterase activity of cutinase variants in two site-saturation libraries relative to TCA assay results. Wild-type enzyme controls are shown as well.

FIGS. 5 and 6 are graphs showing the thermal stability in laundry detergent of cutinase variants in two site-saturation libraries relative to pNB activity results. Wild-type enzyme controls are also shown. These are the same two libraries as FIGS. 4 and 5.

FIG. 7 is a rendition of the 3-dimensional structure of a protein showing the catalytic triad (dark color) active site face of Pseudomonas mendocina cutinase.

FIG. 8 shows the rendition of FIG. 7 and the addition of the initial target amino acids used for the site-saturation mutagenesis procedure of the present invention.

FIG. 9 shows the rendition of FIG. 7 and sites demonstrating at least 2% variants with improved protein activity.

FIG. 10 shows the rendition of FIG. 7 and sites in which the majority of mutations had no effect on the protein activity.

FIG. 11 shows the rendition of FIG. 7 and sites in which the majority of mutations had a detrimental effect on the protein activity.

FIG. 12 shows the rendition of FIG. 7 and sites demonstrating at least 2% of variants with improved protein stability.

FIG. 13 shows the rendition of FIG. 7 and sites in which the majority of mutations had no effect on protein stability.

FIG. 14 shows the rendition of FIG. 7 and sites in which the majority of mutations had a detrimental effect on protein stability.

FIG. 15 shows the activity of a second generation variant versus the wild type.

FIG. 16 shows the increased thermostability of a second generation variant versus the wild type.

FIG. 17 illustrates the DNA sequence for cutinase (SEQ ID NO: 1 ).

FIG. 18 illustrates the amino acid and DNA sequence for cutinase.

### DETAILED DESCRIPTION OF THE INVENTION

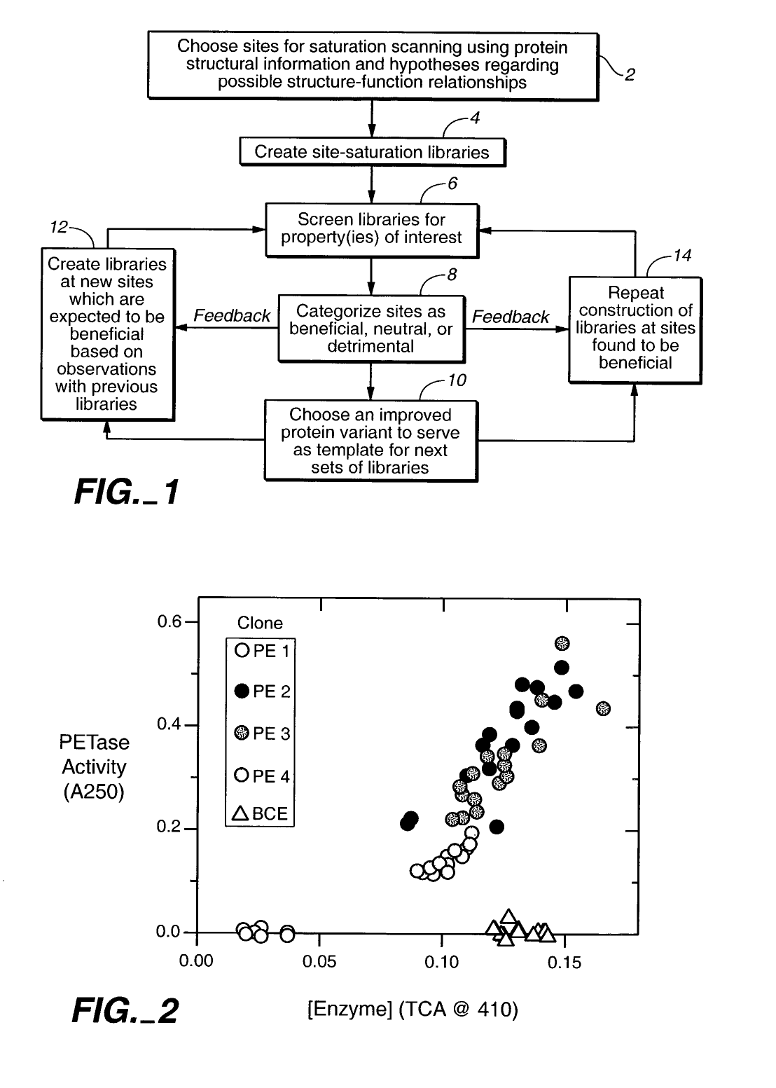

The invention provides a scanning and site-saturation mutagenesis method for producing an improved protein variant using as few as two sequential amino acid substitutions. The method includes feedback adjustment from systematic screening result evaluation for multiple protein properties following amino acid substitution. The method both identifies and grades amino acid substitutions and the mutation sites of the substitutions based upon the results of assays selected to detect at least one desired, improved protein property. The method further correlates the mutation sites and the specific mutations with the assay results to identify trends and enable construction of new molecular libraries and graded identification of mutation sites for further improvement. The method repeats mutagenesis of the initial libraries and results in cooperative mutations that improve protein properties with as few as two mutations. New libraries also are created at new sites selected with grading feedback. The method is set out generally in FIG. 1.

Sites are selected for saturation based upon protein structural information and/or any hypotheses related to structural-functional relationships at step 2, and site-saturation libraries are created at step 4. The created libraries are then screened at step 6 for property(ies) of interest, and the screening results are used to categorize sites at step 8. For instance, the sites are ranked as beneficial, neutral, or detrimental, or other categories are selected as desired. At step 10, an improved variant is selected based on the results of step 8, and the variant is used as a template for additional library construction. Library construction at step 12 utilizes the template variant from step 10 and creates new sites expected to be beneficial based on feedback from the categorized results from step 8. In addition to new libraries, the improved variant from step 10 is used to repeat the previous libraries at the beneficial sites at step 14 using feedback from the categorized results from step 8.

The site-saturation scanning mutagenesis method of the present invention is conducted in an iterative fashion, with feedback adjustment, and can be broken down into the following four sections: site-saturation mutagenesis; activity screening; systematic result evaluation; new library construction/repeat site-saturation mutagenesis of previous libraries.

“Cooperative mutations” means for purposes of this disclosure two or more amino acid mutations.

“Cutinase” means for purposes of this disclosure lipolytic enzymes capable of hydrolyzing cutin substrates. Cutinases are known from various fungi and from bacterial sources. Cutinases of the present invention include those described in P. E. Kolattukudy, “Lipases”, Ed. B Borgstrom and H. L. Brockman, Elsevier 1984, 471-504; S. Longhi et al., J. of Molecular Biology, 268 (4), 779-799 (1997); U.S. Pat. No. 5,827,719; WO 94/14963; WO 94/14964; WO 00/05389; Appl. Environm. Microbiol 64, 2794-2799, 1998; Proteins: Structure, Function and Genentics 26, 442-458, 1996; J. of Computational Chemistry 17, 1783-1803, 1996; Protein Engineering 6, 157-165, 1993. The parent cutinase may have an amino acid sequence which is at least 50% (particularly 60%, 70%, 80%, and 90%) homologous to the cutinase of the bacteria Pseudomonas cutinase SEQ ID NOS: 1 & 2. The cutinase may be naturally occurring or genetically modified cutinase obtained by UV irradiation, N-methyl-N′-nitrosoguanidine (NTG) treatment, ethyl methanesulfonate (EMS) treatment, nitrous acid treatment, acridine treatment or the like, recombinant strains induced by the genetic engineering procedures such as cell fusion and gene recombination and so forth.

“Grading” means for purposes of this disclosure, ranking, rating, or categorizing protein properties, including identifying the properties as beneficial, neutral, and detrimental, and including grouping such ranked properties and marking the groups by color-coding the data and three-dimensional renditions, and including identifying structural-functional trend analysis.

“Homologous or homology” means for purposes of this disclosure any cutinase from any Pseudomonas species, or any other bacterial or fungal source, having a DNA sequence that encodes 50%, 60%, 70%, 80%, or 90% of the DNA SEQ ID NOS: 1 and 2 of the cutinase of Pseudomonas mendocina.

“Molecular Library” means for purposes of this disclosure, a vector-based assembly of at least one nucleic acid molecule that is vector based, non-vector based, or combined vector and non-vector based.

“Mutations” means for purposes of this disclosure, insertions, deletions or duplications to peptide sequences or wild-type nucleic acid sequences, including, but not limited to, point mutations.

“Polyester” means for purposes of this disclosure, aliphilic and aromatic linear polymeric molecules containing in-chain ester groups and which are derived from the condensation of a diacid with a diol or from the polymerization of hydroxyl acids. The principle polyesters in industrial usage include polyethylene terephthalate (PET), tetramethylene terephthalate (PTMT), polybutylene terphthalate (PBT), polytrimethylene terephthalate (PTT), polylactic acid (PLA), and polyethylene naphthalate (PEN), polycyclohexanedimethylene terephthalate (CHDMT), poly(ethylene-4-oxybenzoate A-Tell, polyglycolide, PHBA and 2GN.

“Polyesterase” means for purposes of this disclosure, an enzyme that has significant capability to catalyze the hydrolysis and/or surface modification of polyester. Suitable polyesterases may be isolated from animal, plant, fungal and bacterial sources. The aforementioned microorganisms may be, in addition to being isolated from wild strains, may be isolated from any of mutant strains obtained by UV irradiation, N-methyl-N′-nitrosoguanidine (NTG) treatment, ethyl methanesulfonate (EMS) treatment, nitrous acid treatment, acridine treatment or the like, recombinant strains induced by the genetic engineering procedures such as cell fusion and gene recombination and so forth

“Protein” means for purposes of this disclosure, natural, synthetic, and engineered enzymes such as oxidoreductases, transferases, isomerases, ligases, hydrolases; antibodies; polypeptides; peptides; hormones; cytokines; growth factors; other biological modulators; protein-based diagnostic reagents; and biosensors. Examples of enzymes that may be enhanced by the method of the invention include, but are not limited to, proteases, carbohydrases, lipases, peroxidases, cellulases, and dioxygenases.

“Protein properties” means for purposes of this disclosure protein structural integrity and/or function, including binding and/or catalysis relative to one or more conditions or substrates, protein activity including but not limited to binding and catalysis; protein stability relative to exposure to selected conditions, protein stability including but not limited to thermal stability, pH range stability, ionic strength range stability, pressure stability; reaction substance environment stability, such as stability in organic solvents.

“Protein structural considerations” means for purposes of this disclosure, available information about the physical structure of the protein of interest, including but not limited to substrate binding site location, three-dimensional structure, amino acid sequences; and, the chemical nature of the reaction or binding event to be addressed by mutagenesis of the protein. The use of protein structural considerations is illustrated by the following two examples which are not meant to be limiting. When both the site of substrate binding and the three-dimensional structure are known, site-saturation mutagenesis may be limited to a substrate binding site if the target property is catalytic activity. When only the amino acid sequence of the target protein is known, mutagenesis sites can be selected by comparing the known sequence to homologous sequences to allow selection of suspected functionally important sites for mutagenesis. Both examples further include consideration of the nature of the problem to be solved, i.e. alteration of catalytic activity, binding, stability, etc.

“Pseudomonas” means for purposes of this disclosure any of the members of the bacteria genus Pseudomonas, which are gram negative, rod-shaped bacterium. Examples of the microorganism belonging to the genus Pseudomonas used in the present invention include, for example, P. fluorecens, P. synxantha, P. mendocina, P. synxantha, P. aruginosa, P. aureofaciens. The aforementioned microorganisms may be, in addition to wild strains, any of mutant strains obtained by UV irradiation, N-methyl-N′-nitrosoguanidine (NTG) treatment, ethyl methanesulfonate (EMS) treatment, nitrous acid treatment, acridine treatment or the like, recombinant strains induced by the genetic engineering procedures such as cell fusion and gene recombination and so forth.

“Site-saturation mutagenesis” means for purposes of this disclosure, a method of mutagenesis wherein oligonucleotide primers are randomized at the site of interest using the triplet sequence NN G/C, where N is a mixture of all 4 bases. For each site selected, the amino acid is randomly replaced with all 19 possible alternatives.

“Wild-type” means for purposes of this disclosure, a precursor protein from which a variant is derived.

The method of the invention is divided into the four sections set out below for clarity of presentation and is not meant to be limiting. Those skilled in the art will recognize that other organizational divisions of the method may be used to describe the present invention.

Site-Saturation Mutagenesis

The site-saturation mutagenesis method of the present invention preferably is limited to a selected subset of amino acid positions in the protein defined after a thorough analysis of hypotheses regarding the protein structural and functional relationships.

Following selection of the subset of amino acid positions in the protein for mutagenesis (step 2 in FIG. 2), site-saturation libraries are made at the selected sites by random substitutions of all 19 amino acids using conventional oligonucleotide-directed mutagenesis (step 4 in FIG. 1). Such conventional library formation is well known to those skilled in the art and was carried out generally as described by Airaksinen, A and Hovi, T. (1998) Nucleic Acids Research, Vol.26, No.2: 576-581. Other site-saturation methods may be used and kits, such as the Stratagene Quik-Change™ kit or the Sculptor Mutagenesis Kit (Amersham, UK) will be apparent to those skilled in the art.

The mutagenic oligonucleotide employed is complementary to the areas contiguous to the selected sites. The codon for the selected sites is replaced by the sequence N,N, (G/C mixture). Because the mutagenesis method is not limited to mutations produced by a single codon substitution, the method determines site importance relative to desired protein properties, and identifies the amino-acid substitutions which produce the most efficacious results. The method of the present invention is applicable to a variety of mutagenesis methods.

Activity Screening

Following site-saturation mutagenesis, members of the library undergo screening for the protein properties of interest using appropriate assays for the protein of interest (step 6 in FIG. 1). The screening preferably is conducted for more than one property to provide additional information regarding the quality and mutational effects of the screening data. The probability, P, that a collection of variants y contains at least one clone with a given amino acid is determined from the Poisson distribution formula.

P=1−exp(−np)

p=probability of finding the particular amino acid in the clone mixture

n=the number of clones screened.

For example, if 90 clones are screened, there is a 94% probability of identifying an amino acid coded for by one of the 32 possible codons.

Systematic Result Evaluation

Next, the activity screening results for each variant are evaluated and categorized relative to the position of the mutation in the protein to identify any structure-based trends (step 8 in FIG. 1). Evaluations for trends include, but are not limited to, one or more of the following examples using the assay results: (1) plotting the spatial distribution of variant sites within a three-dimensional rendition of the structure of the protein; and/or (2) plotting the identity of the amino acid mutation; and/or (3) plotting the distribution of beneficial mutation sites compared to amino acid sequences of similar proteins; and/or (4) grading and categorizing results, for example, as beneficial, detrimental, and neutral. The evaluated results, particularly categories, may be color-coded. The color-coding may be added to plotted data and superimposed on known, three-dimensional structural renditions of the protein. Such an analysis produces data that clearly reveals non-uniform site distribution for the various categories, and quickly points out trends for beneficial, detrimental and neutral amino acid substitutions. The analysis and result evaluation feedback enables informed selection of further mutagenesis sites and construction of new libraries.

New Library Construction/Repeat Mutagenesis

The data from the systematic result evaluation is used as feedback to construct new libraries and to select promising mutation sites for the previously tested libraries (steps 12, 14 in FIG. 1). The new libraries at step 14 are constructed using the optimum variants identified as having beneficial mutations following primary mutagenesis. The optimum variants are used as templates for an additional mutagenesis procedure (step 10 in FIG. 1). In this construction method, libraries are made at each of the sites that produced improved or beneficial variants in the previous mutagenesis and screening steps. Following a second site-saturation mutagenesis procedure using as the parent DNA a clone with an amino acid mutation identified as beneficial, the optimum variants will each have two beneficial mutations. The procedure can be repeated until any protein improvement goal is reached, or until the number of beneficial sites is exhausted. Such new library construction essentially repeats previous libraries using an improved starting protein chosen from one of the previous libraries and using identified beneficial mutations.

A second method of constructing a new library utilizes the trend analysis described above to mutate additional sites which are located near any observed and categorized beneficial mutation sites (step 12 in FIG. 1). Step 12 also extends to producing mutations based on trends other than spatial proximity that are observed in beneficial sites. Such library construction extends to new, potentially beneficial amino acid sites, allows exclusion of categorized detrimental sites, and may lead to additional improvements.

### EXAMPLES

The examples are meant to illustrate the method of the present invention and are not intended to be limiting thereof. The examples apply the method of the invention to produce an enzyme having increased stability and increased hydrolytic activity.

### Example 1

Site-Saturation Mutagenesis of Pseudomonas mendocina Cutinase

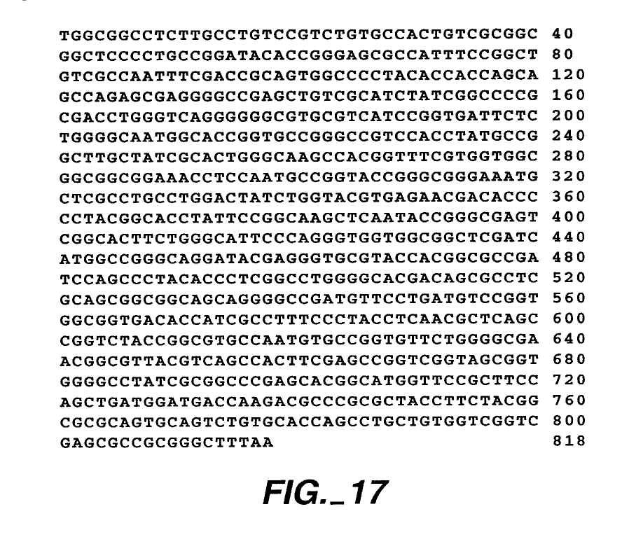

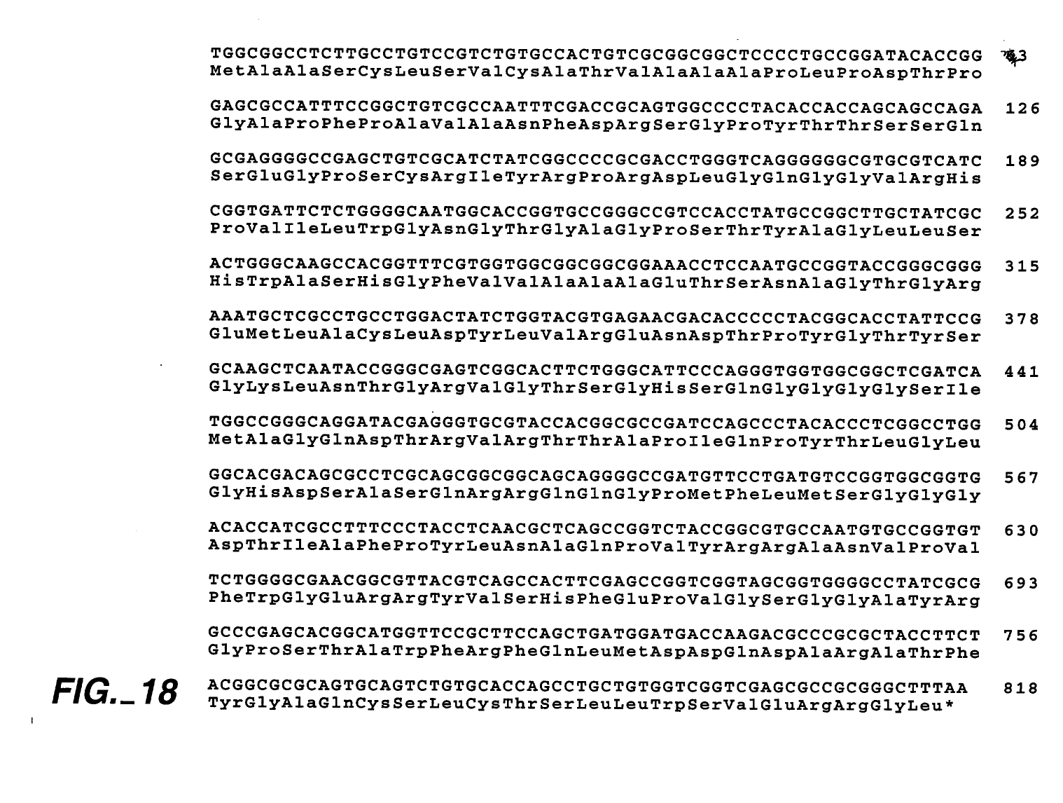

Cutinase from P. mendocina (FIGS. 17 and 18, SEQ ID NO: 1 & 2) was subjected to the method of the invention to identify mutants that improve hydrolytic activity on polyester in poly(ethylene terephthalate) fibers and resin and/or enzyme stability.

The culture media used was buffered by MOPS, which is ( 3-(N-morpholino)propanesulfonic acid. The media also contained 5 μg/mL chloramphenicol to select for transformants harboring the cutinase gene. All chemicals used are obtainable from known scientific reagent suppliers. The culture media was prepared as follows:

MOPS 1A Starter Medium - to prepare 1000 mL (filter sterilize)

| | | |
|---|---|---|
| 100 | mL | 10x MOPS (see below) |
| 12.6 | g | Glucose |
| 0.23 | g | K₂HPO₄ |
| 0.53 | g | NH₄Cl |
| | | Adjust pH to 7.3 with KOH |

MOPS-Urea Medium - to prepare 1000 mL (filter sterilize)

| | | |
|---|---|---|
| 100 | mL | 10x MOPS (see below) |
| 0.52 | g | K₂HPO₄ |
| 3.6 | g | Urea |
| 21 | g | Glucose |
| 35 | g | Maltrin-150 |
| | | Adjust pH to 7.4 with KOH |

10x MOPS - to prepare 1000 mL (filter sterilize)

| | | |
|---|---|---|
| 83.7 | g | MOPS |
| 7.2 | g | Tricine |
| 12.0 | g | KOH |
| 0.48 | g | K₂SO₄ |
| 1.07 | g | MgCl₂.6H₂O |
| 29.2 | g | NaCl |
| 100 | mL | 100x Micronutrients (see below) |

100x Micronutrients - to prepare 1000 mL (filter sterilize)

| | | |
|---|---|---|
| 400 | mg | FeSO₄.7H₂O |
| 100 | mg | MnSO₄.H₂O |
| 100 | mg | ZnSO₄.7H₂O |
| 50 | mg | CuCl₂.2H₂O |
| 100 | mg | CoCl₂.6H₂O |
| 100 | mg | NaMoO₄.2H₂O |
| 100 | mg | Na₂B₄O₇.10H₂O |
| 1.1 | g | CaCl₂ (or 10 mL of 1.0 M stock) |
| 1.47 | g | Sodium Citrate |

Primary Screening Site-Saturation Mutagenesis:

Site-saturation variant libraries were created at amino acid positions corresponding to residue positions 57-66, 68, 85, 86, 88, 125-127, 130, 148-152, 154,155, 176-183, and 204-211 of SEQ ID NO: 2 in P. mendocina cutinase, expressed in Bacillus subtilis strain 3934. The sequence of the parent cutinase is attached hereto as FIG. 17. Libraries were created with the Stratagene Quik-Change™ kit using oligonucleotide primers randomized with NN(G/C) at the target position. Each selected amino acid was randomly replaced with all 19 possible alternatives. Colonies obtained from the variant libraries were selected and placed into 96-well growth plates that contained 100 μL/well of MOPS 1A starter medium. The plates were incubated at 37° C. overnight with continuous humidified shaking at 260 rpm. A sample from each well was replicated into a Millipore 96-well filter plate which contained 200 μL/well of MOPS-Urea medium. 50 μL of 45% glycerol was added to each well of the initial growth plates, which then were stored at −70° C.

The filter plates were incubated for 4 days at 37° C. with continuous humidified shaking at 260 rpm to allow growth and enzyme production. Next, the plates were filtered and assayed for hydrolysis activity using a polyester hydrolysis assay, extracellular cutinase concentration using a TCA turbidity assay, and thermal stability in laundry detergent. Screening was conducted as described below.

Assays

Assay for Hydrolysis Activity on Polyester:

This polyester hydrolysis assay monitors the release of soluble terephthalate-containing fragments resulting from the enzymatic hydrolysis of insoluble polyester (polyethylene terephthalate or PET). The fragments have a significant absorbance at approximately 240-250 nm.

Insoluble PET polyester substrates in the form of fabric samples (Dacron 54 from Testfabrics) or PET resin powder (Scientific Polymer Products catalog # 138, cryogenically ground to <400 μm particle size) were used. The powdered PET samples were placed into a dispensing device (V&P Scientific), to distribute approximately 18 mg uniform samples into the wells of 96-well conventional assay plates. When PET fabric samples were used, ⅝-inch circular pieces of the fabric were cut and placed into 12-well assay plates.

For the PET powder hydrolysis assay, two plates were prepared, one containing PET powder and one with no substrate. Each of the 96 wells of the two assay plates received 200 μL of a reaction buffer containing 50 mM Tris pH 8.6 and 0.01% Brij-35 (#430AG-6 from Sigma). The plate without PET substrate was used as an assay blank.

20 μl of enzyme variant samples were added to an appropriate well in each of the two assay plates. Both assay plates were sealed with plate sealers from Marsh Products. The assay plate containing the PET sample was placed in a covered 40° C. incubator, and the plate was incubated for 18 hours with shaking at 250 rpm. The covered blank assay plate was maintained at room temperature.

Following incubation, 180 μL samples from each well of the PET substrate plate was transferred to a filter plate with a 12-channel pipettor device. The ends of the pipettes were removed by cutting to avoid clogging with the PET powder. Each sample of the PET filter plate was filtered into a well of a new 96-well assay plate.

100 μl from each well of the substrate plate and the blank plate were transferred to a UV-transparent plate, and the absorbance of each sample was determined in a plate reader at 250 nm. The absorbance of the wells from the blank plate without substrate was subtracted from the corresponding wells in the assay plate with PET substrate.

TCA Assay to Determine Cutinase Concentration:

20 μL of the filtered culture supernatant was added to each well of a 96-well flat bottom plate which contained 100 μL/well of purified water. Light scattering/absorbance was determined at 410 nm using 5 second mixing mode in a plate reader to determine base-line blank values. 100 μL of 15% w/v TCA (trichloro-acetic acid) was added to each well prior to incubating the plate for between 5 and 30 minutes at room temperature. Light scattering/absorbance caused by protein precipitation was determined at 410 nm using a plate reader set for 5 second mixing mode.

The results were calculated by subtracting the blank reading, which did not contain TCA, from the sample readings with TCA. The light scattering/absorbance results were linear with respect to the enzyme concentrations present in the samples, thereby allowing direct plotting of concentration against enzyme performance as shown in FIG. 2. FIG. 2 shows the results obtained from the untransformed host strain (PE-1), three wild-type cutinase transformants (PE-2 through PE-4) and one negative control, transformed with a cellulase gene (BCE). Good correlation is observed between polyester hydrolysis activity and the TCA signal. Plots of this nature allow identification and selection of variants with elevated hydrolytic activity.

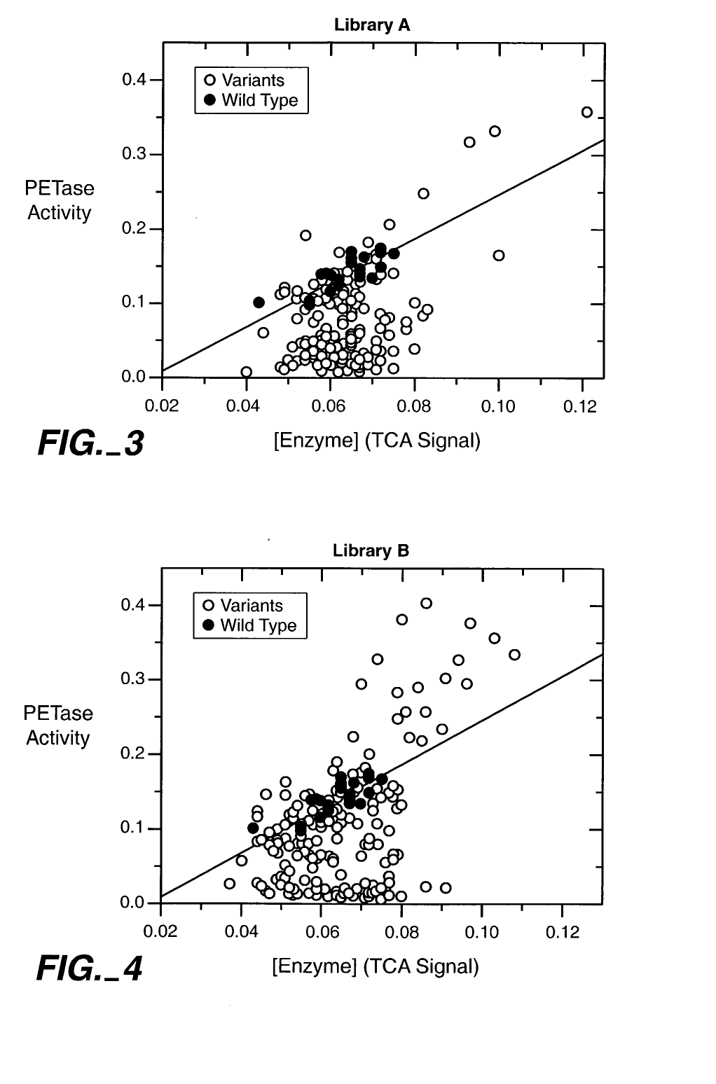

FIGS. 3 and 4 show data from two site-saturation libraries, at amino-acid positions Leu 154 (Library A) and Phe 180 (Library B). It can be seen that the wild type controls fall along the same activity-concentration line; the line shown is the best fit to these data points by linear regression. It can also be seen that the variants fall above, below, and along this line, signifying better, worse, or similar specific activity relative to the wild-type controls. It can also be seen that the library at position 180 exhibits more diversity and more improved variants than the library at position 154.

Enzyme Stability Assay:

Stability assays were conducted to determine thermostability of the variants in a detergent medium. Stability of the variants obtained from the site-saturation mutagenesis procedure was determined by adding 10 μL of the filter plate samples into each well of two 96-well plates containing 200 μL/well of liquid detergent (1.6 g/L, 6.3 gpg hardness). The 2 plates were mixed, and one plate was maintained on ice or in an ice-water bath (unstressed plate) while the second plate was incubated in a 40° C. water bath for 10 minutes. (thermostressed plate). The stressed plate was transferred to the ice-water bath, and esterase activity assays were conducted on both plates as described below. Wild type cutinase samples also were run as above for comparison with the variants.

The enzyme activity assay is based upon the cleavage of colorless p-nitrophenyl butyrate (pNB) with esterase in the presence of water to produce butyrate and yellow colored p-nitrophenolate. The assay was conducted using an assay buffer containing 100 mM Tris pH 8.0 and 0.1% (v/v) Triton X-100. The substrate stock was 100 mM p-nitrophenyl butyrate in DMSO, prepared by adding 174.3 μL pNB to 10 mL DMSO. The substrate stock was divided into 1-mL aliquots and stored at −20° C.

The pNB assay procedure was as follows: 400 μL of substrate stock was diluted into 40 mL assay buffer for each pair of assay plates. 200 μL of this diluted substrate solution was added to each well of conventional 96-well plates. The plates were equilibrated at 25° C., and the reaction was initiated by adding 10 μL of the enzyme/detergent solution from a stressed or unstressed plate. The plate was mixed and inserted into the plate reader and data acquisition was begun. The activity rate (mAU₄₁₀/minute) was determined. The pNB reaction rate is proportional to the concentration of active enzyme. For each well, the fraction of active enzyme after stress was calculated using the following formula:

Fraction active=(stressed pNB activity-blank)/(unstressed pNB activity-blank).

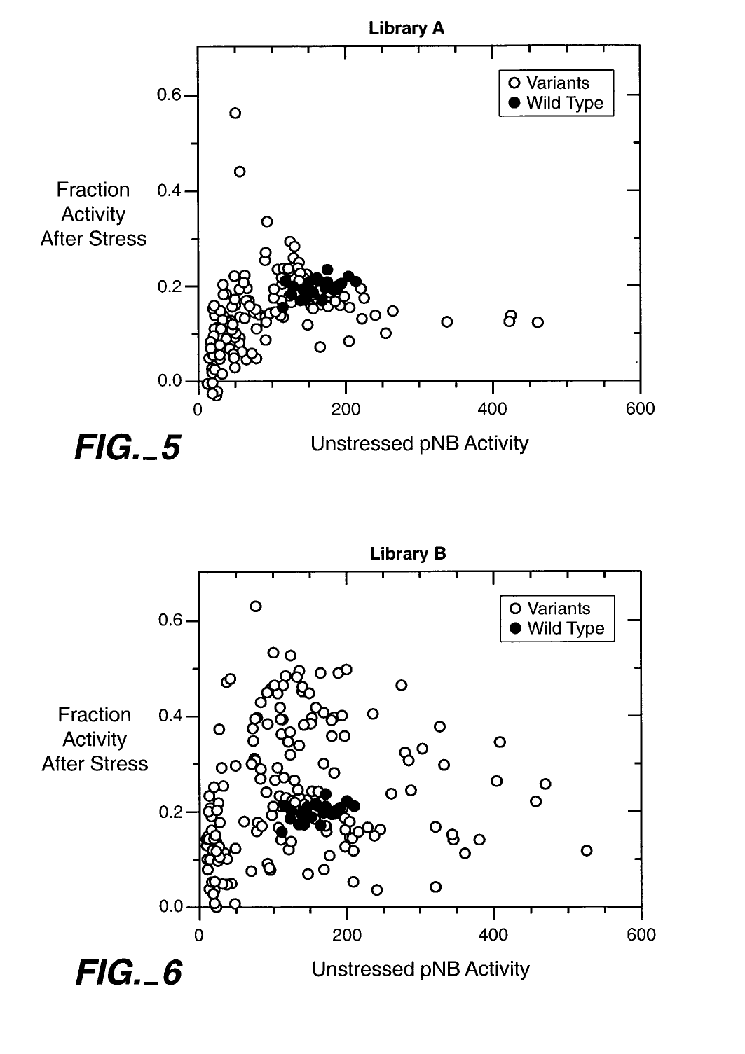

The fraction of active enzyme after stress was plotted against the pNB activity of the unstressed enzyme for each variant, as shown in FIGS. 5 and 6 which depict the results for site-saturation libraries at amino acid positions Leu 154 (Library A) and Phe 180 Library B). The FIGS. 5 and 6 results show thermostable variants having unstressed pNB activity, and also identify some variants which have apparently elevated thermostability only because the pNB activity is too low to produce an accurate ratio. FIGS. 5 and 6 further demonstrate that mutations to different sites produce different enzymatic properties. For instance, the library shown in FIG. 5 produced only a few promising variants with activity greater than the wild-types, while the library shown in FIG. 6 produced a number of variants with considerably greater activity than the wild-types.

Systematic Result Evaluations

The results from the TCA assay, the PET hydrolysis assay, and the stability assay were calculated, using conventional, known formulas as set out above, using Excel™ software. Variants were graded and ranked for stability and hydrolysis activity. Table 1 shows the results obtained from the site-saturation mutagenesis procedure described in this example. The results were graded and color coded as follows: green=more than 2% of the variants at the listed sites had significantly greater activity than the control (beneficial mutation); blue=the majority of the variants had unchanged activity (neutral mutation); red=the majority of the variants had significantly decreased activity relative to the control (detrimental mutation); and brown=approximately half of the variants had unchanged activity and half had decreased activity (neutral mutation). The color coding was repeated using the same colors and same percentage criteria for stability results. As can be seen in Table 1, color coding easily identifies those variants having both desired enzyme properties.

[image]

Secondary screening to repeat the previous libraries and create new libraries was conducted using the variants having beneficial properties as shown in Table 1. The frozen glycerol stock samples for each improved variant were streaked onto appropriate media and incubated to obtain single colonies. The colonies from each variant were inoculated into tubes containing 3 mL LB medium and shaken at 37° C. for 6-18 hours.

800 μL of broth from each incubated tube was combined with 400 μL of 45% glycerol and stored at −70° C. One mL of broth was centrifuged, the supernatant was removed, and the pellet was stored at −20° C. for DNA sequencing.

100 μL of each broth was pipetted in multiple replicates into a 96-well plate. A sample from each well was replicated into a filter plate containing 200 μL/well of MOPS-Urea medium. The filter plates were allowed to grow for 4 days at 37° C. with humidified shaking at 260 rpm. The plates were filtered and the TCA, PET hydrolysis, and stability assays described above were conducted. The results were calculated as described above, and the samples of the highest performing variants were pooled. Enzyme concentration of the pooled sample was determined by SDS-PAGE using densitometry or LC/MS using an ¹⁵N-labeled wild-type cutinase standard. The variants were tested for activity on polyester fabric in buffer and in detergent. The results are shown in Table 2, which lists the site and substitution made for variants with elevated stability, and in Table 3 which lists variants with improved hydrolysis of polyester (PET). The F180-S205 last seven variants shown in Table 2 represent a second library as created in step 14 of FIG. 1 showing two beneficial mutations which greatly increased relative stability of these seven variants. A number of variants with two mutations also are shown in Table 3.

TABLE 2
Polyesterase Variants with Elevated Stability

| | | Secondary Screen | | |
|---|---|---|---|---|
| | | Variant | Wild Type | Stability Relative |
| Clone Number | Mutations | Stability | Stability | To Wild Type |
| PE1-2-G4 | F180I | 0.707 | 0.428 | 1.65 |
| PE2-1-C11 | F180L | 0.809 | 0.428 | 1.89 |
| PE3-2-E3 | F180N | 0.709 | 0.428 | 1.66 |
| PE4-2-A12 | F180P | 0.696 | 0.428 | 1.63 |
| PE5-2-E5 | G59F | 0.556 | 0.276 | 2.02 |
| PE6-1-H5 | G59K | 0.514 | 0.196 | 2.62 |
| PE7-1-G5 | G59L | 0.545 | 0.196 | 2.78 |
| PE8-1-H1 | G59V | 0.626 | 0.196 | 3.19 |
| PE9-1-D5 | S205G | 0.819 | 0.332 | 2.47 |
| PE10-2-E5 | S20R | 0.651 | 0.472 | 1.38 |
| PE11-2-A7 | S63R | 0.646 | 0.290 | 2.23 |
| PE12-1-C9 | S85H | 0.457 | 0.196 | 2.33 |
| PE13-1-D9 | S85K | 0.502 | 0.196 | 2.56 |
| PE14-2-E6 | T177H | 0.748 | 0.304 | 2.46 |
| PE15-2-D5 | T177L | 0.813 | 0.304 | 2.68 |
| PE16-2-C7 | T177Y | 0.717 | 0.304 | 2.36 |
| PE17-2-F11 | T64A | 0.536 | 0.237 | 2.26 |
| PE18-1-E8 | T64L | 0.624 | 0.237 | 2.63 |
| PE19-1-F6 | Y150F | 0.620 | 0.332 | 1.87 |
| PE20-2-E8 | Y182A | 0.662 | 0.290 | 2.28 |
| PE21-2-D2 | Y182L | 0.622 | 0.290 | 2.14 |
| PE229-2-C5 | Y182P | 0.933 | 0.290 | 3.21 |
| PE23-2-E12 | F180A-S205G | 0.530 | 0.063 | 8.35 |
| PE24-2-C6 | F180H-S205G | 0.836 | 0.063 | 13.17 |
| PE25-2-A4 | F180K-S205G | 0.644 | 0.063 | 10.14 |
| PE26-2-G3 | F180L-S205G | 0.561 | 0.063 | 8.84 |
| PE27-1-D2 | F180N-S205G | 0.735 | 0.063 | 11.58 |
| PE28-2-A2 | F180P-S205G | 0.670 | 0.063 | 10.56 |
| PE29-2-E9 | F180S-S205G | 0.461 | 0.063 | 7.27 |

TABLE 3
Polyesterase Variants and Performance Summary

| | | Tertiary Screen | | | | | |
|---|---|---|---|---|---|---|---|
| | | Polyesterase | Wild Type | Relative | Polyesterase | Wild Type | Relative |
| | | activity | activity | buffer activity | activity | activity | detergent activity |
| Clone Number | Mutations | in buffer | in buffer | of variant | in detergent | in detergent | of variant |
| PE308-1-C2 | A66E | 1.219 | 0.867 | 1.41 | 0.052 | 0.043 | 1.22 |
| PE23-2-E12 | F180A-S205G | 0.827 | 0.632 | 1.31 | 0.118 | 0.051 | 2.31 |
| PE24-2-C6 | F180H-S205G | 0.742 | 0.632 | 1.18 | 0.089 | 0.051 | 1.75 |
| PE31-2-G4 | F180I | 2.677 | 1.210 | 2.21 | 0.306 | 0.137 | 2.24 |
| PE32-2-G1 | F180K-S205G | 0.639 | 0.632 | 1.01 | 0.146 | 0.051 | 2.87 |
| PE26-2-G3 | F180L-S205G | 0.747 | 0.632 | 1.18 | 0.099 | 0.051 | 1.95 |
| PE3-2-E3 | F180N | 2.142 | 1.210 | 1.77 | 0.250 | 0.137 | 1.83 |
| PE27-1-D2 | F180N-S205G | 0.782 | 0.632 | 1.24 | 0.103 | 0.051 | 2.02 |
| PE28-2-A2 | F180P-S205G | 1.033 | 0.632 | 1.64 | 0.175 | 0.051 | 3.43 |
| PE29-2-E9 | F180S-S205G | 0.971 | 0.632 | 1.54 | 0.134 | 0.051 | 2.62 |
| PE5-2-E5 | G59F | 0.671 | 0.594 | 1.13 | 0.061 | 0.056 | 1.09 |
| PE33-2-E10 | G59L | 0.703 | 0.594 | 1.18 | 0.072 | 0.056 | 1.30 |
| PE34-2-D8 | G61D | 1.099 | 0.594 | 1.85 | 0.115 | 0.056 | 2.07 |
| PE35-2-D10 | G61E | 1.029 | 0.594 | 1.73 | 0.120 | 0.056 | 2.16 |
| PE36-1-B9 | I178M-F180V- | 1.306 | 0.632 | 2.07 | 0.153 | 0.051 | 2.99 |
| | S205G | | | | | | |
| PE37-1-G1 | R40Q-Y112Y | 1.609 | 1.210 | 1.33 | 0.150 | 0.137 | 1.10 |
| PE38-1-C1 | S205N-F207L | 0.992 | 1.210 | 0.82 | 0.157 | 0.137 | 1.15 |
| PE11-2-A7 | S63R | 0.571 | 0.738 | 0.77 | 0.046 | 0.041 | 1.12 |
| PE13-1-D9 | S85K | 1.025 | 0.867 | 1.18 | 0.067 | 0.043 | 1.58 |
| PE14-2-E6 | T177H | 0.964 | 0.594 | 1.62 | 0.141 | 0.056 | 2.55 |
| PE15-2-D5 | T177L | 0.891 | 0.594 | 1.50 | 0.124 | 0.056 | 2.24 |
| PE16-2-C7 | T177Y | 0.936 | 0.594 | 1.58 | 0.151 | 0.056 | 2.73 |
| PE18-1-E8 | T64L | 0.599 | 0.462 | 1.30 | 0.070 | 0.056 | 1.27 |
| PE20-2-E8 | Y182A | 0.947 | 0.738 | 1.28 | 0.064 | 0.041 | 1.57 |
| PE22-2-C5 | Y182P | 0.893 | 0.738 | 1.21 | 0.057 | 0.041 | 1.38 |

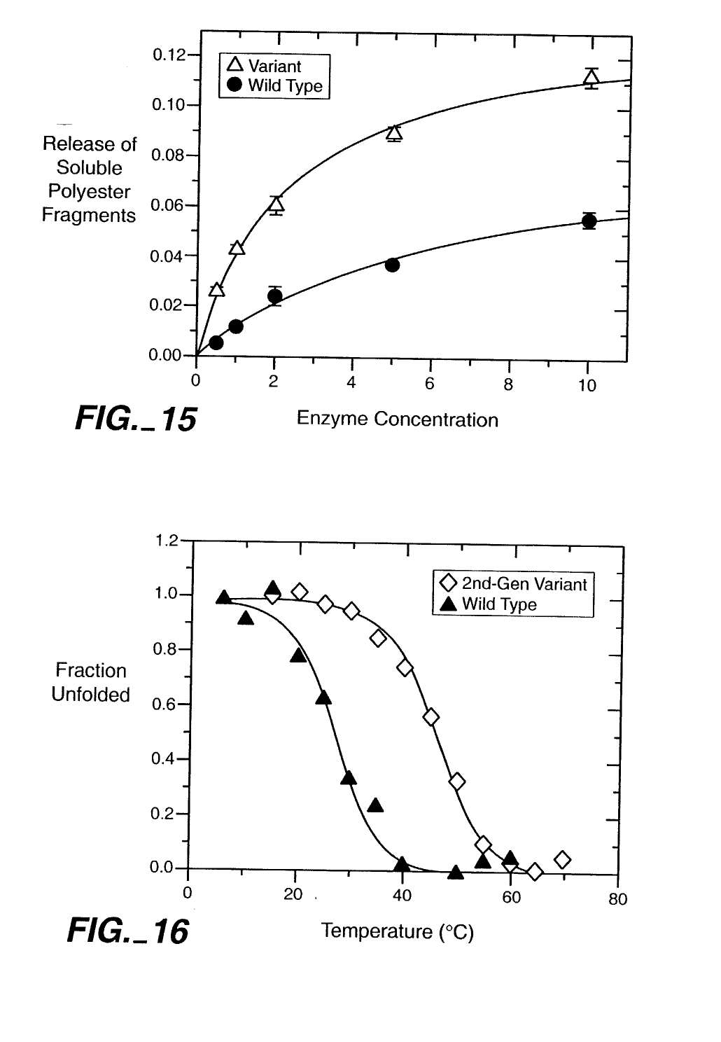

The improvements in the properties of the second generation variants when tested in a detergent environment are shown graphically in FIGS. 15 and 16. FIG. 15 demonstrates that the second generation variant has a 3-fold increase in activity as compared to the wild type when plotted against enzyme concentration. FIG. 16 shows that the thermostability of the second generation variant is greatly increased as compared to the wild type.

A summary of the results of the method of the present invention includes the initial screening procedure results of Table 1, which establishes a list of sites where mutations produced variants having at least some polyesterase activity and some stability. Additionally, Table 1 shows that mutations at the following sites produced variants having more than two percent of the clones with greater activity than controls: Sites 63 -65, 178-182, 205, 209, and 211. Mutations at the following sites resulted in variants having more than two percent of the clones with greater stability than controls: 59, 64, 150, 177, 180, and 182. The following sites represent variants with both improved activity and stability: 64, 180, 182, and 211. Clones were selected based upon the promising sites shown in Table 1, and secondary screening was conducted by repeating the previous libraries and by creating new libraries at new sites expected to be beneficial.

Tables 2 and 3 represent, respectively, secondary stability screening steps and tertiary activity screening steps using the selected clones identified during primary screening as templates. Tables 2 and 3 identify the mutations made at the selected sites and include combinations of mutations. The substitutions of serine at locations 20 and 85 represent the creation of new libraries at new sites which are expected to be beneficial.

Table 2 shows that substitution of phenylalanine at site 80 with isoleucine, leucine, asparagines, or proline resulted in variants having improved stability as compared to the wild-type; substitution of glycine at site 59 with phylalanine, lysine, leucine, or valine produced variants with improved stability as compared to the wild type. Substitutions of phenylalanine at site 180 in combination with substitutions of glycine at site 205 resulted in variants with significantly increased relative stability.

As can be seen in table 3, specific substitutions at one to three sites produced variants with improved polyesterase activity.

Structural Context Evaluation

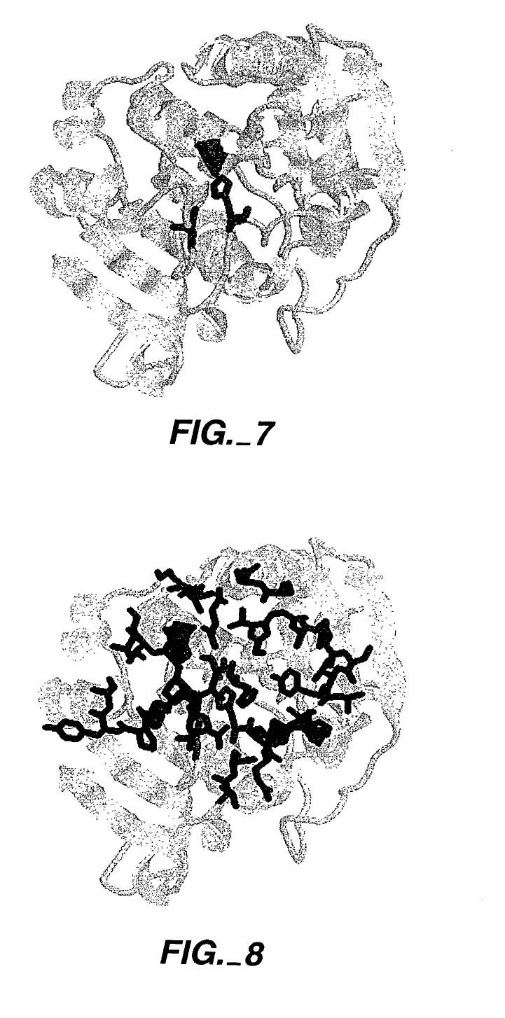

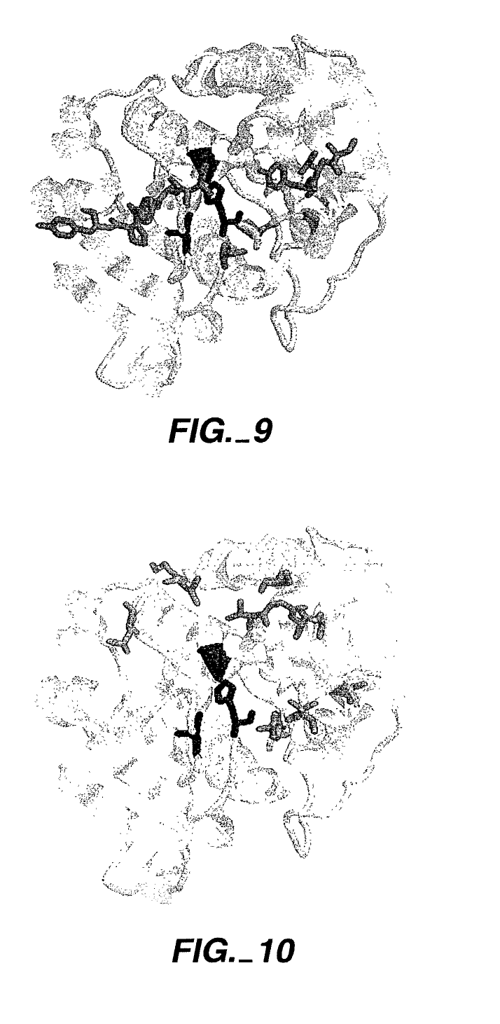

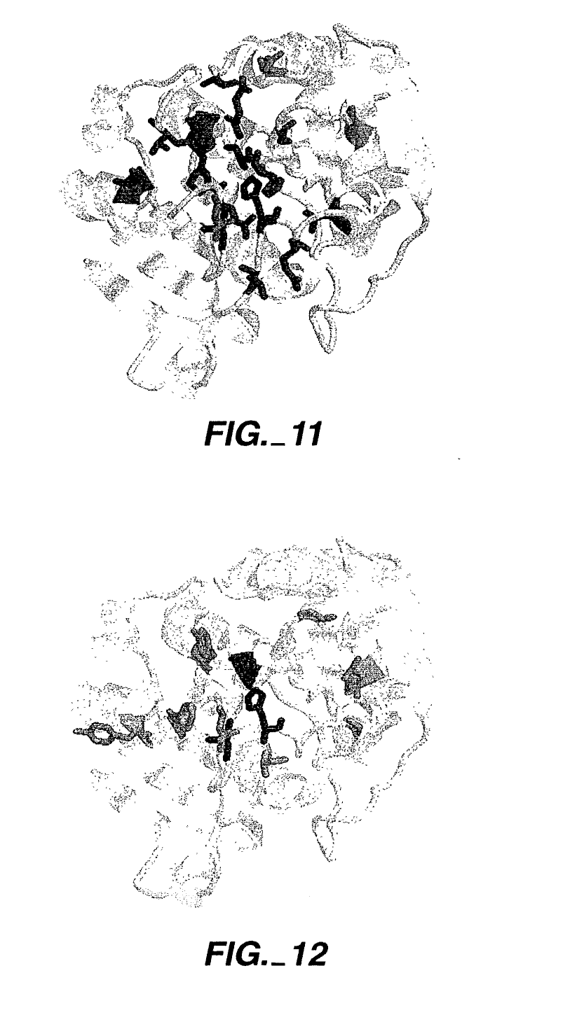

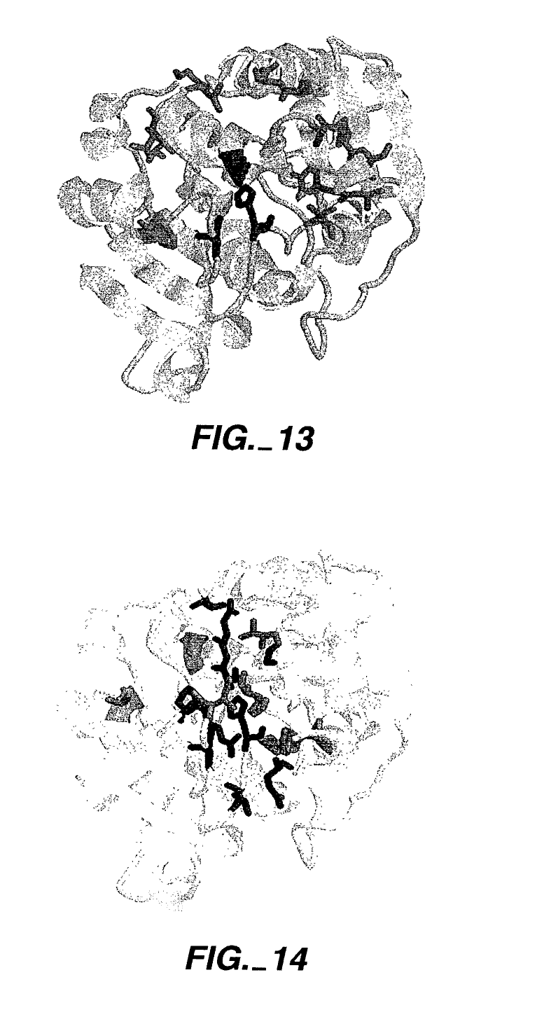

The graded screening results (improvement, detriment, or neutral) were examined in the context of position within the protein to elucidate any underlying structurally based trends. The results are shown in bold superimposed on maps of the structure of the protein, as shown in FIGS. 7-14. The results also were color-coded (not shown) using the same colors used in Table 1. FIG. 7 shows in bold the catalytic triad active site face of the Pseudomonas mendocina cutinase. (Ser 126, Asp 176, His 206). FIG. 8 shows in bold the addition of the initial target amino acids that were used for the site-saturation scanning mutagenesis procedure. FIG. 9 shows in bold the sites which resulted in at least 2% of the variants having significantly improved polyester hydrolysis activity. FIG. 10 shows in bold the variant sites in which the majority of mutations had no effect on activity. FIG. 11 shows in bold the variant sites in which the majority of mutations generally had a detrimental effect on activity. FIG. 12 shows in bold the variant sites which resulted in a least 2% of the variants having significantly improved stability. FIG. 13 shows in bold the variant sites in which the majority of mutations generally had no effect on stability. FIG. 14 shows in bold the variant sites in which the majority of mutations generally had a detrimental effect on stability.

FIGS. 7-14 related the variants to structure thereby suggesting further modification sites. For instance, in the examples shown, detrimental sites tend to be buried in both the activity and stability maps and confined to generally the same locations. Improvement sites for activity and for stability, tend to occupy similar locations within the structure of the protein. Sites with no effect also occupy similar positions within the protein structure for both activity and stability results.

The method of the present invention may be used to engineer proteins with enhanced properties for use in fields other than textile modifications as described above. Such fields include, but are not limited to, healthcare, foods and feed, brewing, cosmetics, agriculture, textiles, detergents, environmental waste conversion, biopulp processing, biomass conversion to fuel, and other chemical procedures.

The examples above are not meant to be limiting, and the invention is intended to apply to mutagenesis of any protein. For those proteins where three dimensional maps are not available and only the amino acid sequence is known, the method may be carried out by comparing the known sequence to other sequences which are homologous. Using this method, dissimilar sites or sites with suspected functional importance are mutated because non-conserved sites generally are more readily available to accommodate amino acid substitutions.

The scope of the invention is described in the following claims.

What is claimed is:

1. A method for obtaining a variant having one or more desired protein properties comprising: selecting amino acid sites in a protein for mutation; performing mutagenesis at the selected mutation sites to create a library; screening the library for variants having one or more desired protein properties; grading the mutation sites of the variants for the one or more desired protein properties; selecting one or more variants having a desirable grade as a template for, with feedback from the grading, creating and screening additional libraries, whereby the method utilizes cooperative mutations to obtained a variant having at least two mutations.

2. The method of claim 1 wherein said mutagenesis is performed by site-saturation mutagenesis and wherein selecting amino acid sites is performed by utilizing protein structural considerations.

3. The method of claim 2 wherein creating and screening additional libraries is performed by repeating site-saturation mutagenesis at mutation sites having desirable grades and performing site-saturation mutagenesis at new sites on new libraries.

4. The method of claim 3 wherein performing site-saturation at new sites is performed by selecting sites located near mutation sites having desirable grades.

5. The method of claim 2 wherein the protein structural considerations are binding site location, three-dimensional structure, amino acid sequence, nature of chemical reaction, or nature of chemical binding.

6. The method of claim 1 wherein the protein property is an enzyme property.

7. The method of claim 6 wherein the enzyme property is one or more of catalysis, binding, or stability.

8. The method of claim 1 wherein the screening for one or more variants is performed by selecting and conducting appropriate assays for the one or more protein properties of interest.

9. The method of claim 1 wherein grading is performed by identifying trends.

10. The method of claim 9 wherein identifying trends is performed by plotting a spatial distribution of graded sites on a three-dimensional rendition of the protein.

11. The method of claim 9 wherein identifying trends is performed by plotting amino acid mutation identities.

12. The method of claim 9 wherein identifying trends is performed by plotting a distribution of graded mutation sites.

13. The method of claim 2 wherein creating and screening additional libraries is performed by screening the additional libraries for the desired protein properties and repeating site-saturation mutagenesis until a desired protein property goal is attained.

14. A method for obtaining a variant enzyme having one or more desired properties comprising: selecting amino acid sites utilizing a three-dimensional rendition of the enzyme; performing site-saturation mutagenesis at the selected mutation sites to create a library; screening the library for variants having one or more desired properties; grading the mutation sites of the variants for the one or more desired properties; selecting one or more variants having a desirable grade as a template; using the template and feedback to repeat site-saturation mutagenesis at mutation sites having desirable grades and to perform site-saturation mutagenesis on new libraries at new sites.

15. The method of claim 14 wherein the one or more desired properties are substrate activity, thermostability, stability relative to reaction environment, ionic strength range of stability, pressure stability, or pH range of stability.

16. The method of claim 14 wherein the one or more desired properties is substrate activity and thermostability.

17. The method of claim 14 wherein the enzyme is cutinase.

18. A process for the production of a cutinase variant with hydrolytic activity on polyester, the cutinase from Pseudomonas species, the process comprising: utilizing a three-dimensional model to select for mutation amino acid sites likely to demonstrate hydrolytic activity; performing site-saturation mutagenensis at the selected mutation sites on a library; screening the library for variants using assays to detect polyesterase activity and thermostability; grading the mutated sites as beneficial, neutral or detrimental for both polyesterase activity and thermostability; selecting a variant having at least one beneficial grade; creating new and repeat libraries using the selected variant and feedback from the grading.
